(19) United States
(12) Patent Application Publication
(10) Pub. No.: US 2011/0184450 A1
(43) Pub. Date: Jul. 28, 2011

(54) ANEURYSM EMBOLIZATION DEVICE

(76) Inventor: Norikata Takuma, Tokyo (JP)

(21) Appl. No.: 13/010,000

(22) Filed: Jan. 20, 2011

(30) Foreign Application Priority Data
Jan. 22, 2010 (JP) 12055/2010

(51) Int. Cl. A61M 29/00

(52) U.S. Cl. 606/194

(57) ABSTRACT

An aneurysm embolization device includes at least: a sac-shaped balloon dome part which is inserted into an aneurysm in a blood vessel, then expanded and left therein; and a balloon plane part which is provided at an opening of the balloon dome part and covers an aneurysm mouth part. The balloon plane part includes: a first hole and a second hole communicating with the inside and the outside of the balloon dome part, respectively. The balloon dome part includes a gap forming member which forms a non-contact region where, when the balloon dome part is expanded in the aneurysm, an outer surface of the balloon dome part does not come into contact with an inner surface of the aneurysm. When the aneurysm embolization device is placed and deployed in an aneurysm having a blood vessel branching therefrom, a gap is formed between the outer surface of the balloon dome part and the inner surface of the aneurysm. Thus, a treatment for the aneurysm can be carried out without occluding an entrance of the branched blood vessel.

### BACKGROUND OF THE INVENTION

1. Field of the Invention

The present invention relates to an aneurysm embolization device for treating an aneurysm in a blood vessel by occluding the aneurysm. Specifically, the present invention relates to techniques allowing a treatment for an aneurysm to be carried out even when a blood vessel branches from the aneurysm itself.

2. Description of the Related Art

An aneurysm is local dilatation of a blood vessel due to weakening of the vascular wall, and usually has a size of 3 mm to 4 mm, or sometimes of 15 mm or larger in diameter. If untreated, an aneurysm can increase in size, and eventually ruptures. The rupturing of the aneurysm is likely to cause fatal bleeding: for example, bleeding onto the brain surface (subarachnoid hemorrhage). A clipping method and intravascular surgical techniques are known for treating such aneurysms.

The clipping method is a surgical technique that blocks blood from flowing into an aneurysm and eliminates the aneurysm from blood flow with a pinching member called a clip, which is made of an elastic, corrosion-resisting material such as titanium. Specifically, in the method, the aneurysm is exposed by craniotomy, and a neck part (the base) of the aneurysm is pinched with the clip for the blockage and elimination in order to prevent the aneurysm from rupturing (see, for example, Japanese Examined Patent Application Publication No. Hei 07-004389).

Meanwhile, intravascular surgical techniques include balloon placement, coagulant injection, coil embolization and stent placement techniques.

The balloon placement is a technique to occlude an aneurysm with a balloon using a catheter. The balloon, which is made of latex or silicone rubber and connected to a tip of the catheter, is guided to an aneurysm, inserted thereinto and then expanded therein. Then, the balloon is detached from the catheter. In other words, this is a technique to place a balloon within an aneurysm so as to occlude a sac and neck of the aneurysm while maintaining blood flow in a parent blood vessel (artery) (see, for example, Japanese Patent Translation Publication No. 2004-520881 and “Nokekkan-nai chiryo no Do's & Don'ts (Do's & Don'ts in intracerebrovascular treatment)” pp. 1-4, published by Igaku-Shoin Ltd.).

The coagulant injection is a technique to inject a liquid coagulating substance (i.e., liquid embolic material) inside an aneurysm (see, for example, Japanese Unexamined Patent Application Publication No. Hei 06-107549 and “Nokekkan-nai chiryo no Do's & Don'ts (Do's & Don'ts in intracerebrovascular treatment)” pp. 1-4, published by Igaku-Shoin Ltd.).

The coil embolization is a technique in which: a catheter is percutaneously introduced into a blood vessel from, for example, a femoral region; a tip of the catheter is moved to an aneurysm site under radiographic guidance; and an embolus, such as a platinum coil, is supplied from a lumen formed in the catheter and fills (packs) the inside of the aneurysm (see, for example, Japanese Patent Application Publication No. 2003-070794 and “Nokekkan-nai chiryo no Do's & Don'ts (Do's & Don'ts in intracerebrovascular treatment)” pp. 1-4, published by Igaku-Shoin Ltd.).

The stent placement is a technique to block blood from flowing into an aneurysm from a parent blood vessel with a tubular member called a stent. The stent is left at a mouth part of the aneurysm and radially expands at a narrow part of the vessel from the inside of the blood vessel to cover the aneurysm (see, for example, Japanese Patent Application Publication No. 2004-33535).

There are also other techniques: to inject a coagulating substance into an aneurysm through slits arranged in a peripheral body part of a stent; and to segregate an aneurysm in the blood vessel from blood flow by covering the mouth part of the aneurysm using the stent as a scaffold (see, for example, Japanese Patent No. 4057318).

All of such aneurysm treatments provide reduction in the blood flowing into the aneurysm and result in blockage of blood flow and formation of a clot. In other words, these techniques can treat aneurysms based on pathophysiological characteristics of blood: i.e., blood coagulates when its flow stops; abnormal blood vessel inner wall or contact between blood and foreign substances causes formation of blood clot; and the like.

However, in the clipping method as represented by the invention described in Japanese Examined Patent Application Publication No. Hei 07-004389, it is difficult to pinch a neck part of an aneurysm with a clip when the neck part is wide (i.e., when the mouth part of the aneurysm has a large diameter). In addition, a lot of preparation tasks are required for the craniotomy because it is a major operation as compared with an intravascular operation. Furthermore, the operation may compress or damage the brain tissue.

In the balloon placement technique as represented by the invention described in Japanese Patent Translation Publication No. 2004-520881, a balloon which is tied to a catheter using a rubber string is prepared. Then, the balloon is detached from the catheter or a joint between the balloon and the catheter is separated using a high-frequency electric current so that the balloon may not be deflated. However, it is very difficult to keep the balloon from deflating after the balloon is detached from the catheter, and it is also required to keep watch on the balloon not to deflate even after the separation. In addition, the shape of the neck part of aneurysms greatly differs one from the others, but the shape of balloons is virtually the same. As a result, the balloon is not able to occlude the aneurysm completely because the shape of the neck part of aneurysm and that of the balloon do not always match (i.e., the balloon is not able to correspond to various shapes of aneurysms).

In the coagulant injection as represented by the invention described in Japanese Unexamined Patent Application Publication No. Hei 06-107549, selection of a liquid substance to fill an aneurysm is difficult, because the liquid substance must be liquid until filled into the aneurysm and must promptly coagulate right after filled into the aneurysm. In other words, if the coagulation rate of the liquid substance is too fast, the substance coagulates in a catheter or the like before it is filled into an aneurysm. Meanwhile, if the coagulation rate of the liquid substance is too slow, the liquid substance filled in the aneurysm would flow out into a blood vessel.

Besides, the coil embolization as represented by the invention described in Japanese Patent Application Publication No. 2003-070794 overcomes the problems of the coagulant injection and balloon placement. However, packing of an aneurysm with a coil is difficult in the coil embolization, and there is also a risk of bleeding by breaking the aneurysm wall during a process of the operation. Furthermore, there are risks that a part of the coil may stick out into the parent blood vessel, or that the coil packed in the aneurysm may escape into the parent blood vessel if the neck part of the aneurysm is wide. Still furthermore, even if the coil is packed into the aneurysm completely, approximately half a space in the aneurysm remains unfilled. The space may allow blood to flow in the aneurysm and thus embolization could be failed in some cases.

In addition, the stent placement as represented by the inventions described in Japanese Patent Application Publication No. 2004-33535 and Japanese Patent No. 4057318 has a risk to block a way in other necessary branched blood vessel as well as the aneurysm if the blood vessel branches around the site of the aneurysm.

Against this background, the applicant has already proposed an aneurysm embolization device capable of occluding an aneurysm readily and reliably in an appropriate manner according to an onset site of the aneurysm, the shape of a neck part of the aneurysm, and the size thereof. FIGS. 18 and 19 show the aneurysm embolization device.

This aneurysm embolization device 100 includes: a sac-shaped balloon dome part 1 which is inserted into an aneurysm in a blood vessel, then expanded and left therein; and a balloon plane part 2 which is provided at an opening of the balloon dome part 1 and covers a mouth part of the aneurysm. The balloon plane part 2 has a hole 12 communicating with the inside of the balloon dome part 1 (see Japanese Patent Nos. 4312255 and 4334611). Accordingly, when the balloon dome part 1 is inserted into an aneurysm An and the aneurysm embolization device 100 is arranged so that the balloon plane part 2 can be left on the mouth part of the aneurysm An, the balloon plane part 2 can cover a desired region around the aneurysm An as well as the mouth part of the aneurysm An regardless of the shape and the size of the mouth part of the aneurysm An. Moreover, the hole 12 of the balloon plane part 2 allows blood flowing in a parent blood vessel By to flow into the balloon dome part 1. Then, blood BL2 flowed in the balloon dome part 1 causes the balloon dome part 1 to expand, and pushes blood which is already in the aneurysm An (i.e., a small amount of blood BL1 left at the inner wall of the aneurysm An and the outside of the balloon dome part 1) out of the aneurysm, that is, to the parent blood vessel. Thereafter, the stream of the blood BL2 which flowed into the balloon dome part 1 is repressed by covering and closing of the aneurysm with the balloon plane part 2, thereby forming a clot. Thus, the aneurysm An can be readily and reliably occluded.

However, even the aneurysm embolization devices disclosed in Japanese Patent Nos. 4312255 and 4334611 may not be able to cope with a situation where there is a branched blood vessel inside an aneurysm.

Specifically, for an aneurysm An having a branched blood vessel bv, for example, as shown in FIG. 20A, the aneurysm An needs to be occluded while the blood flowing inside the parent blood vessel Bv can securely flow into the branched blood vessel bv. However, when the aneurysm embolization device 100 is placed in the aneurysm An as shown in FIG. 20B, there are risks as follows. Specifically, the balloon plane part 2 may occlude the entrance to the aneurysm An, and block the flow of the blood flowing from the parent blood vessel Bv through the aneurysm An into the branched blood vessel by. Additionally, the balloon dome part 1, which is expanded inside the aneurysm An by the blood flowing from the parent blood vessel Bv, may occlude the entrance to the branched blood vessel bv, resulting in blocking the blood flow.

### SUMMARY OF THE INVENTION

The present invention addresses the problems discussed above. An object of the present invention is to provide an aneurysm embolization device capable of occluding an aneurysm readily and reliably in an appropriate manner according to an onset site of the aneurysm, the shape of a neck part of the aneurysm, and the size thereof, and also capable of treating an aneurysm, even when a blood vessel branches from the aneurysm itself, without occluding an entrance of the branched blood vessel.

An aneurysm embolization device according to a first aspect of the present invention is an aneurysm embolization device for treating an aneurysm in a blood vessel by occluding the aneurysm. The aneurysm embolization device includes at least: a sac-shaped balloon dome part which is inserted into the aneurysm, then expanded and left therein; and a flexible sheet-shaped balloon plane part which is provided at an opening of the balloon dome part and covers a mouth part of the aneurysm. In the aneurysm embolization device, the balloon plane part includes: a first hole communicating with an inside of the balloon dome part; and a second hole communicating with an outside of the balloon dome part, and the balloon dome part includes a gap forming member which forms a desired non-contact space, where an outer surface of the balloon dome part does not come into contact with an inner surface of the aneurysm, when the balloon dome part is expanded in the aneurysm.

An aneurysm embolization device according to a second aspect of the present invention is the aneurysm embolization device according to the first aspect, in which a diameter of the opening of the balloon dome part is larger than a diameter of the first hole of the balloon plane part.

An aneurysm embolization device according to a third aspect of the present invention is the aneurysm embolization device according to any one of the first and second aspect, in which the balloon plane part further includes an anchor member (stent) which is expanded and left in the blood vessel.

An aneurysm embolization device according to a fourth aspect of the present invention is the aneurysm embolization device according to the third aspect, in which the anchor member is attached to at least one end of the balloon plane part.

An aneurysm embolization device according to a fifth aspect of the present invention is the aneurysm embolization device according to any one of the first to fourth aspects, in which the gap forming member is a plurality of convex bodies protruding on the outer surface of the balloon dome part.

An aneurysm embolization device according to a sixth aspect of the present invention is the aneurysm embolization device according to the fifth aspect, in which the convex bodies are granular bodies arranged at desired intervals.

An aneurysm embolization device according to a seventh aspect of the present invention is the aneurysm embolization device according to the fifth aspect, in which the convex bodies are at least two wall-shaped bodies arranged substantially parallel to each other at a desired interval.

An aneurysm embolization device according to an eighth aspect of the present invention is the aneurysm embolization device according to any one of the first to fourth aspects, in which the gap forming member is a convex body having a through hole formed on the outer surface of the balloon dome part.

An aneurysm embolization device according to a ninth aspect of the present invention is the aneurysm embolization device according to the eighth aspect, in which the convex body is a tubular body having a hollow part therein.

An aneurysm embolization device according to a tenth aspect of the present invention is the aneurysm embolization device according to the eighth aspect, in which the convex body is a U-shaped body forming a hollow part with the outer surface of the balloon dome part.

The aneurysm embolization device according to the present invention includes: a sac-shaped balloon dome part which is inserted into an aneurysm in a blood vessel, then expanded and left therein; and a flexible sheet-shaped balloon plane part which is provided at an opening of the balloon dome part and has a hole communicating with the inside of the balloon dome part. The balloon plane part includes: a first hole communicating with the inside of the balloon dome part; and a second hole communicating with the outside of the balloon dome part. In addition, the balloon dome part includes a gap forming member which forms a non-contact region where, when the balloon dome part expands in the aneurysm, the outer surface of the balloon dome part does not come into contact with the inner surface of the aneurysm.

Accordingly, when the aneurysm embolization device is placed so that the balloon dome part can be inserted into an aneurysm having a blood vessel branching from the aneurysm itself, the balloon plane part is left on the mouth part of the aneurysm. Therefore, the balloon plane part can cover a desired region around the aneurysm as well as the mouth part and regardless of the shape and size of the mouth part. Moreover, the first hole provided to the balloon plane part allows blood flowing inside a parent blood vessel to flow into the balloon dome part. In this event, when the balloon dome part is expanded by the blood flowing in through the first hole, the gap forming member provided to the balloon dome part serves as a spacer for forming a partial gap between the outer surface of the balloon dome part and the inner surface of the aneurysm. This gap formed outside the balloon dome part and from the second hole of the balloon plane part to the branched blood vessel prevents the balloon dome part from occluding the entrance to the branched blood vessel.

As a result, the stream of the blood flowed into the balloon dome part is repressed by covering and closing the aneurysm with the balloon plane part, and thus, the blood in the balloon dome part forms a clot to occlude the aneurysm. In addition, because of the clot formation by the blood in the balloon dome part, the balloon dome part prevents the aneurysm embolization device from being swept away by the blood flow, thus functioning as an anchor member to aid the remaining of the device at the site of aneurysm. Furthermore, blood from the parent blood vessel flows into the gap formed between the outer surface of the balloon dome part and the inner surface of the aneurysm through the second hole of the balloon plane part. Then, the gap guides the blood to the branched blood vessel. Accordingly, the blood flowing inside the parent blood vessel flows into the branched blood vessel without being blocked. Thus, the blood flow can be secured. In this case, the blood flow is secured the most in the shortest distance between the second hole and the branched blood vessel, whereas, at a position far away from this blood flowing portion, the blood flow stagnates and a clot is formed. Especially, at a position inside the aneurysm which is on an opposite side of the part where the branched blood vessel starts, the blood flow stagnates, resulting in an early-stage development of blood clot.

Thus, it is possible to provide an aneurysm embolization device capable of occluding an aneurysm readily and reliably in an appropriate manner according to an onset site of the aneurysm, the shape of a neck part of the aneurysm, and the size thereof, and also capable of treating an aneurysm, even when a blood vessel branches from the aneurysm itself, without occluding the entrance to the branched blood vessel.

### BRIEF DESCRIPTION OF THE DRAWINGS

FIG. 1 is an entire perspective view showing an aneurysm embolization device according to the present invention.

FIG. 2 is a side view of the aneurysm embolization device shown in FIG. 1, showing a side where a gap forming member is formed.

FIG. 3 is a sectional view of the aneurysm embolization device shown in FIG. 2, taken along the line I-I.

FIG. 4 is a partially-enlarged horizontal sectional view for illustrating a structure of the gap forming member provided to the aneurysm embolization device according to the present invention and a non-contact region formed by the gap forming member.

FIG. 5 is a longitudinal sectional view showing another aneurysm embolization device according to the present invention.

FIG. 6 is a longitudinal sectional view showing another aneurysm embolization device according to the present invention.

FIG. 7 is a longitudinal sectional view showing another aneurysm embolization device according to the present invention.

FIG. 8 is a side view for illustrating another gap forming member provided to the aneurysm embolization device according to the present invention.

FIG. 9 is a partially-enlarged horizontal sectional view for illustrating a structure of a gap forming member of the aneurysm embolization device shown in FIG. 7 and a non-contact region formed by the gap forming member.

FIG. 10 is a side view for illustrating another gap forming member provided to the aneurysm embolization device according to the present invention.

FIG. 11 is a partially-enlarged horizontal sectional view for illustrating a structure of the gap forming member of the aneurysm embolization device shown in FIG. 10 and a non-contact region formed by the gap forming member.

FIG. 12 is a partially-enlarged horizontal sectional view for illustrating another structure of the gap forming member of the aneurysm embolization device shown in FIG. 10 and a non-contact region formed by the gap forming member.

FIG. 13 is a side view for illustrating another gap forming member provided to the aneurysm embolization device according to the present invention.

FIGS. 14A and 14B are process sectional drawings for sequentially illustrating a method for operating the aneurysm embolization device according to the present invention shown in FIG. 6.

FIGS. 15A and 15B are process sectional drawings for sequentially illustrating a method for operating the aneurysm embolization device according to the present invention shown in FIG. 6.

FIGS. 16A and 16B are process sectional drawings for sequentially illustrating a method for operating the aneurysm embolization device according to the present invention shown in FIG. 6.

FIGS. 17A and 17B are process sectional drawings for sequentially illustrating a method for operating the aneurysm embolization device according to the present invention shown in FIG. 6.

FIG. 18 is an entire perspective view showing a structure of a conventional aneurysm embolization device.

FIG. 19 is a sectional view for illustrating an method for operating the conventional aneurysm embolization device.

FIGS. 20A and 20B are sectional views for illustrating inconveniences when the conventional aneurysm embolization device is used.

### DETAILED DESCRIPTION OF THE INVENTION

Hereinafter, examples of embodiments of the present invention will be described with reference to the drawings.

Note that the following embodiments of the present invention have various technological restrictions because these are specific preferable examples of the present invention. However, the scope of the present invention is not limited to these embodiments unless it is otherwise particularly described to be limited in the following explanations.

An aneurysm embolization device intended in the present invention is an aneurysm embolization device to treat an aneurysm in a blood vessel by occluding the aneurysm. Especially, even when there is a branched blood vessel (arteriole) inside the aneurysm, the aneurysm embolization device is capable of occluding an aneurysm readily and reliably in an appropriate manner according to the shape of a neck part of the aneurysm and the size thereof, while allowing a treatment for the aneurysm to be carried out without occluding the entrance to the branched blood vessel.

### Embodiment 1

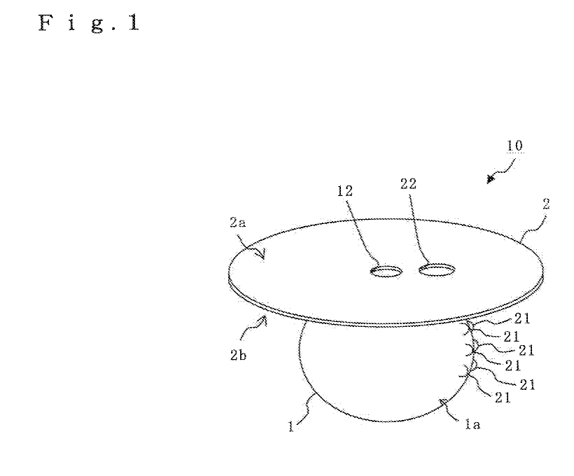

As shown in FIGS. 1 to 3, the aneurysm embolization device 10 of the present embodiment includes a balloon dome part 1 and a balloon plane part 2. Incidentally, FIG. 1 is shown schematically with slight exaggeration to facilitate the understanding of functions of the aneurysm embolization device of the present invention (including functions of both the balloon dome part and the balloon plane part).

The balloon dome part 1 is a sac body, which is inserted into an aneurysm, expanded by influx of blood, and left in the aneurysm. This balloon dome part 1 is made of a flexible, stretchable and thin biocompatible material using, for example, latex or silicone rubber. Additionally, it is desirable that the entire outside diameter of the balloon dome part 1 be almost equal to or slightly smaller than the inside diameter of the aneurysm.

The balloon dome part 1 includes gap forming members 21. The gap forming members 21 have a function of forming a desired non-contact space where, when the balloon dome part 1 expands in an aneurysm, an outer surface la of the balloon dome part 1 does not come into contact with an inner surface of the aneurysm. Here, the desired non-contact space refers to a gap which can sufficiently allow blood flowing inside a parent blood vessel to sufficiently flow into a branched blood vessel.

The gap forming members 21 are constituted of multiple convex bodies that each protrude from the outer surface 1 a of the balloon dome part 1. The gap forming members 21 may be formed integrally with the balloon dome part 1 using the same material as that of the balloon dome part 1, or may be formed separately from the balloon dome part 1 and then attached thereto by bonding or the like. Alternatively, the gap forming members 21 may be formed separately from the balloon dome part 1 using a different material from that of the balloon dome part 1 and then attached thereto by bonding or the like.

The gap forming members 21 are provided in such a manner that, when the balloon dome part 1 is placed and expanded in the aneurysm, a guide path is formed from the vicinity of a second hole 22 (described later) to an entrance of a branched blood vessel formed in the aneurysm. FIGS. 1 to 3 illustrate each of the gap forming members 21 as a granular body formed by partial protrusion of the outer surface 1a of the balloon dome part 1. The granular bodies are arranged at desired intervals. Specifically, shown are multiple (two) of the granular bodies that are respectively arranged right and left at a desired interval, and moreover sets of such two granular bodies are placed vertically in multiple columns (four columns).

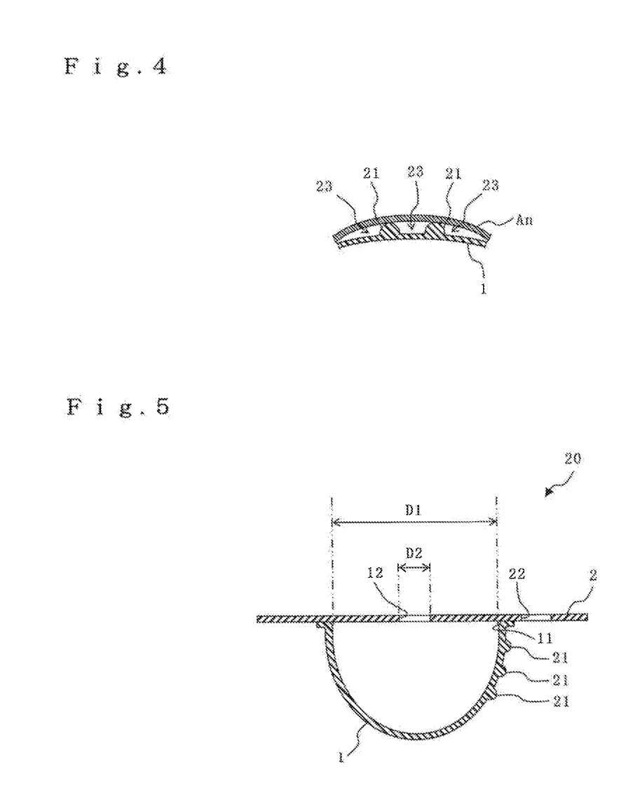

Thus, the desired non-contact space formed by the gap forming members 21 constituted of the partially protruding granular bodies as described above refers to a gap formed around the gap forming members 21, the gap being formed by the gap forming members 21 protruding from the outer surface of the balloon dome part 1 which come into contact with the inner surface of the aneurysm when the balloon dome part 1 expands in the aneurysm. Specifically, the desired non-contact space refers to gaps 23 formed around these gap forming members 21, the gaps 23 mainly including a gap 23 formed between the outer surface of the balloon dome part 1 and the inner surface of the aneurysm An and between two of the gap forming members 21, 21 arranged side-by-side, as shown in FIG. 4. Accordingly, in the aneurysm embolization device 10 shown in FIGS. 1 to 3, a gap extending along the outer surface 1a of the balloon dome part 1 is formed as a desired non-contact space by disposing the two gap forming members 21 arranged right and left in four columns.

Note that the shape, arrangement, and the like of the gap forming members 21 are not limited to those described above. The gap forming members 21 can be arbitrarily designed and altered in consideration of the easiness of forming the desired non-contact space, the site of the branched blood vessel formed in the aneurysm, and the like. Other forms of the gap forming members 21 will be described later.

The balloon plane part 2 is a sheet body which is provided at an opening 11 of the balloon dome part 1 and covers a mouth part of the aneurysm. This balloon plane part 2 is made of a flexible thin biocompatible material. In FIG. 1, the balloon plane part 2 is indicated as a round shape, but it is not limited to this shape. The balloon plane part 2 may be oval, rectangular, or the like, and can be arbitrarily designed or selected depending on the shape and size of a mouth part of the aneurysm to be occluded. Accordingly, it is desirable that size of the balloon plane part 2 be larger than the diameter of the mouth part of the aneurysm.

In this aneurysm-mouth-part closing film 2, it is desirable that one surface 2a of the film be formed from a hard amorphous film such as diamond-like carbon, and that another surface 2b on the opposite side from the one surface 2a have a rough surface like cilia. Such a structure makes clot formation hardly occur on the one surface 2a side of the aneurysm-mouth-part closing film 2; meanwhile, clot formation is likely to occur on the other surface 2b side of the film 2. Therefore, the aneurysm embolization device 10 is placed in a manner that the one surface 2a of the aneurysm-mouth-part closing film 2 faces a parent vessel, and that the other surface 2b faces an aneurysm.

The balloon plane part 2 includes an influx hole 12 serving as a first hole communicating with the inside of the balloon dome part 1, and a flux hole 22 serving as a second hole communicating with the outside balloon dome part 1.

As long as the influx hole 12 allows blood to flow into the balloon dome part 1, the shape of the influx hole 12 is not particularly limited. The balloon plane part 2 may have an opening from the beginning, or a slit hole opened by flux of blood. In FIGS. 1, the influx hole 12 is shown as a circular opening which is opened originally. Although the hole size of the influx hole 12 in diameter is not specified particularly, the size is large enough to allow blood to flow into the balloon dome part 1, and is small enough to prevent clots solidified in the balloon dome part 1 from flowing out.

Likewise, as long as the flux hole 22 allows blood to flow outside the balloon dome part 1, the shape of the flux hole 22 is not particularly limited. The balloon plane part 2 may have an opening from the beginning, or a slit hole to be opened by flux of blood. In FIG. 1, the flux hole 22 is shown as a circular opening which is opened in the first place.

Incidentally, although the hole size of the flux hole 22 is not specified particularly, the size should be large enough to secure the blood stream without preventing blood flowing inside the parent blood vessel from flowing into the branched blood vessel. The number of the flux hole 22 is not limited to one, and multiple, for example 2 to 4, of the flux holes 22 may be provided. Furthermore, the flux hole 22 is desirably provided as close as possible to the opening 11, since this facilitates the blood flowing in between the outer surface of the balloon dome part 1 and the inner surface of the aneurysm.

As long as the balloon dome part 1 and the aneurysm-mouth-part closing film 2 are integrated each other, other features are not particularly limited. For example, these parts may be integrally formed using the same material, or may be formed separately and then integrated together. In addition, integration of the aneurysm-mouth-part closing film 2 to the opening 11 of the balloon dome part 1 may be accomplished by joining the dome part 1 to the film 2 using an adhesive, or may be accomplished by suturing or fusion bonding.

According to the aneurysm embolization device 10 with the above-described constitution, when the aneurysm embolization device 10 is placed so that the balloon dome part 1 can be inserted into an aneurysm having a branched blood vessel therein, the balloon plane part 2 on the one surface 2a side is pushed by blood flow streaming in a parent vessel, meanwhile, the other surface 2b is pressed against the inner blood vessel wall. Thus, the balloon plane part 2 is left on a mouth part of the aneurysm. Accordingly, the balloon plane part 2 covers the mouth part of the aneurysm and a desired area around the aneurysm from inside of the parent vessel. Furthermore, when the balloon plane part 2 covers the mouth part of the aneurysm, blood streaming in the parent vessel flows into the balloon dome part 1 through the influx hole 12 arranged in the balloon plane part 2, expands the balloon dome part 1, and fills the inside of the balloon dome part 1.

In this occlusion process, even when the blood flowing in through the influx hole 12 causes the balloon dome part 1 to expand, the gap forming members 21 provided to the balloon dome part 1 form the gaps 23 between the balloon dome part 1 and the aneurysm. Furthermore, the balloon plane part 2 includes the flux hole 22 that allows blood to flow out of the balloon dome part 1. Thus, blood from the parent blood vessel flows into the gaps 23 formed between the outer surface of the balloon dome part 1 and the inner surface of the aneurysm through the flux hole 22 of the balloon plane part 2. Then, the blood flowed into the gaps 23 is guided to the entrance to the branched blood vessel through the guide path, that is, a series of gaps 23 formed by the multiple gap forming members 21. Thus, the blood flowing inside the parent blood vessel flows into the branched blood vessel without being blocked. In this manner, the blood flow can be ensured.

Then, the stream of the blood flowed into the balloon dome part 1 is repressed, resulting in the formation of blood clot which occludes the aneurysm. Therefore, by using embolization device 10 with a very simple constitution, it is possible to occlude an aneurysm readily and reliably in an appropriate manner according to an onset site of the aneurysm, the shape of a neck part of the aneurysm, and the size thereof, and to treat an aneurysm, even when a blood vessel branches from the aneurysm itself, without occluding the entrance to the branched blood vessel.

In this occlusion process, when the size of the blood clot formed in the balloon dome part 1 is larger than that of the mouth part of the aneurysm, the clotted blood is locked at an edge of the aneurysm mouth part. This makes it possible to prevent the balloon dome part 1 from being easily detached from the aneurysm for a long period. Furthermore, it is desired that surface treatment be performed on the inner and outer surfaces of the balloon dome part 1 to stimulate clot formation.

### Embodiment 2

In the present invention, the influx of blood into the balloon dome part 1 can be ensured. In other words, the diameter of the opening 11 is a size that at least allows influx of blood from the influx hole 12 of the balloon plane part 2 in the above-described balloon dome part 1 of the first embodiment. However, if the diameter of the opening 11 is small, the opening 11 may be twisted during operation of the aneurysm embolization device; as a result, there is a risk to block influx of blood into the balloon dome part 1. Therefore, a third embodiment of the present invention is different from the above-described first embodiment in the structure of an opening 11 of a balloon dome part 1.

Note that, in other embodiments to be described below, description will be given mainly of aspects which are different from those in the above-described first embodiment of the present invention. Therefore, the same letters or numerals are given to constituents which are similar to those in the first embodiment, and explanations thereof are omitted. The constituents represented by the same letters or numerals will be the same unless it is otherwise particularly described.

An aneurysm embolization device of the present embodiment can be exemplified as shown in FIG. 5.

As shown in FIG. 5, an aneurysm embolization device of the present embodiment includes the balloon dome part 1 and the balloon plane part 2. The balloon plane part 2 includes the influx hole 12 and the flux hole 22. The balloon dome part 1 includes the gap forming members 21. Furthermore, a diameter D1 of the opening 11 of the balloon dome part 1 is larger than a diameter D2 of the influx hole 12 of the balloon plane part 2 (D1>D2).

According to the aneurysm embolization device 20 with the above-described constitution, the opening 11 of the balloon dome part 1 can be protected from excess distortion which occurs during operation. Thus, blood is able to flow into the balloon dome part 1 through the influx hole 12 of the balloon plane part 2 via the opening 11 of the balloon dome part 1 reliably. Furthermore, the aneurysm embolization device 40 according to the present embodiment can adequately deal with an aneurysm, for example, which has a hemispherical shape with such a wide neck part that it is difficult to clip (to pinch) the neck part or which has such a shape that packed coils easily escape from the aneurysm. Therefore, it is preferable to use the aneurysm embolization device 40 of the present embodiment as a fundamental tool for such a type of aneurysm which is exerted from a side surface of a blood vessel.

### Embodiment 3

In the present invention, an aneurysm embolization device can be left in a blood vessel more stably. In other words, a third embodiment of the present invention is different from the above-described first and second embodiments in that the third embodiment includes means for stabilizing the balloon plane part 2 in a blood vessel.

Note that, in other embodiments described below, description will be given by taking, as an example, the balloon dome part 1 having a structure with the opening 11 described in the second embodiment.

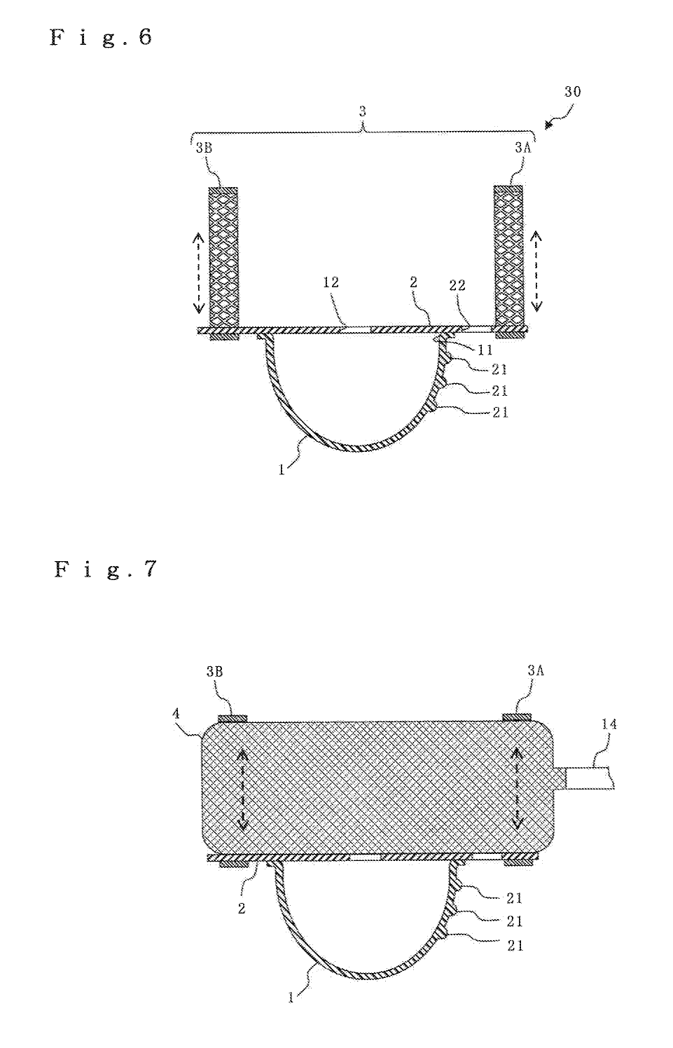

An aneurysm embolization device of the present embodiment including mean for stabilizing the device in a blood vessel can be exemplified as shown in FIG. 6. The aneurysm embolization device shown here has a beneficial effect on usage for a type of aneurysm which is exerted from a side surface of a blood vessel, for example.

As shown in FIG. 6, an aneurysm embolization device 30 of the present embodiment includes the balloon dome part 1 and the balloon plane part 2. The balloon plane part 2 includes the influx hole 12 and the flux hole 22. The balloon dome part 1 includes the gap forming members 21. The balloon plane part 2 further includes an anchor member 3 which is expanded and left in a blood vessel. The anchor member 3 can be stabilized in a blood vessel without displacement, and can be, for example, an approximately tubular stent with a net-like structure. This anchor member (hereinafter, referred to as “stent”) 3 can be formed using a material for medical application such as stainless steel, tantalum, cobalt base alloy and nickel-titanium alloy (nitinol).

Furthermore, as long as the stent 3 is attached to the balloon plane part 2 in an integrated manner, the way of the attachment is not particularly limited. Therefore, for example, these components may be jointed using an adhesive, or the stent 3 may be rolled up, pinched or sutured by the balloon plane part 2.

In FIG. 6, the stent 3 is constituted of two approximately tubular (annular) body cells 3A and 3B, which are separated from each other with the influx hole 12 in between and attached to the respective opposed ends of the balloon plane part 2. The cells can radially expand toward the blood vessel wall as shown with dotted arrows in the figure. The stent 3 (3A and 3B) can be stabilized in a blood vessel without displacement by pressing the stent 3 to the blood vessel wall.

According to the aneurysm embolization device 30 with the above-described constitution, the balloon plane part 2 can be reliably placed adjacent to the mouth part of the aneurysm by expanding the stent 3 in a blood vessel and pressing the stent 3 to the blood vessel wall. Thus, the aneurysm embolization device 30 can be stably left at the mouth part of the aneurysm.

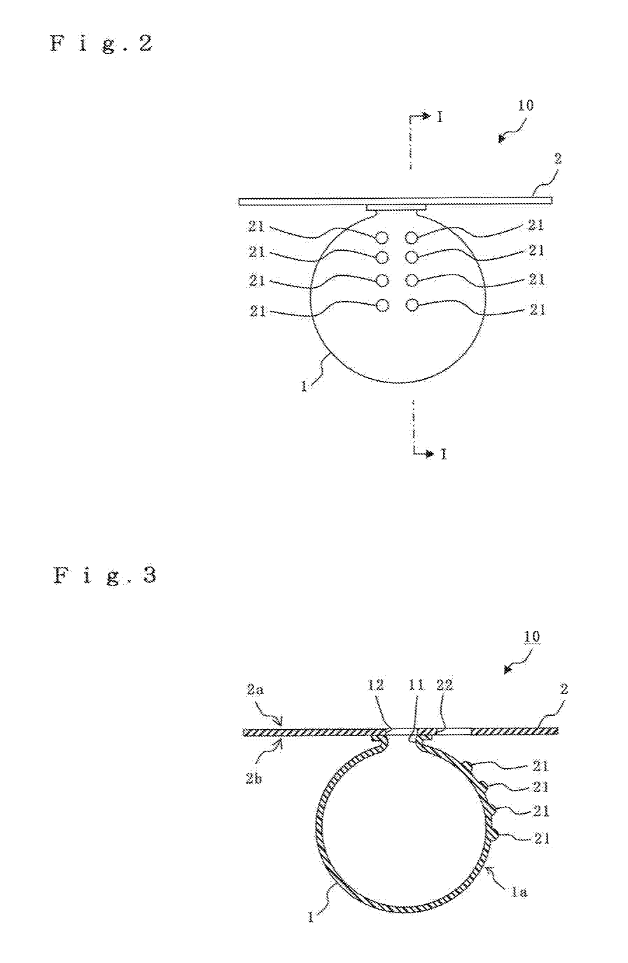

It is also desirable that the stent 3 (3A and 3B) of the above-described third embodiment be attached to at least one end of the balloon plane part 2. The term “one end” refers to a side of a blood vessel which is closer to the heart than an aneurysm, in other words, an upstream side of a blood flow. Therefore, only any one of the two approximately tubular (annular) body cells 3A and 3B shown in FIG. 2 should be provided as the stent 3.

As mentioned above, despite such a simple structure, another end of the balloon plane part 2 deploys along the bloodstream and can ensure stable placement of the aneurysm embolization device in the blood vessel without displacement.

Moreover, in the present embodiment, the means for stabilizing the balloon plane part 2 to a blood vessel is not limited to the above-described approximately tubular stent. For example, although not illustrated, a coiled stent formed of a spirally and approximately tubular body can also be used. This stent can also be formed using a material for medical application such as stainless steel, tantalum, cobalt alloy, and nickel-titanium alloy (nitinol).

Furthermore, when the present invention includes the means for stabilizing the balloon plane part 2 in a blood vessel through the radial expansion, it is desirable that the means more effectively expand radially. In other words, the above-mentioned approximately tubular stent 3 shown in the third embodiment should autonomously expand with its elastic force at a desirable position. However, if not, and if the stent is sought to expand at a desirable position, additional means is needed to support the expansion of the means for stabilizing the balloon plane part 2 in a blood vessel. Therefore, in the present embodiment, the expansion supporting means is further provided.

The aneurysm embolization device including the expansion supporting means of the present embodiment can be exemplified in FIG. 7.

As shown in FIG. 7, the aneurysm embolization device of the present embodiment further includes the expansion balloon 4 which is expandable inside the stent 3. The expansion balloon 4 can push and expand the stent 3 from the inside of the stent 13 by inflating, and can be, for example, a flexible and stretchable bag made of latex, silicone rubber, or the like.

In FIG. 7, the expansion balloon 4 is constituted so that the balloon 4 is inserted and placed into the stent 3 constituted of the approximately tubular body cells 3A and 3B, and that the balloon 4 can be evenly inflated by slowly introducing a liquid, such as isotonic saline, through a liquid-supplying tube 14 thereinto.

According to the aneurysm embolization device with the above-described constitution, radial expansion of the stent 3 in a blood vessel is supported, and the balloon plane part 2 can be stabilized in the blood vessel efficiently and effectively.

### Embodiment 4

Additionally, in the present invention, the gap forming members can take other forms. Specifically, in the above-described embodiments, the gap forming members are partially protruding granular bodies, but are not limited thereto in the present invention.

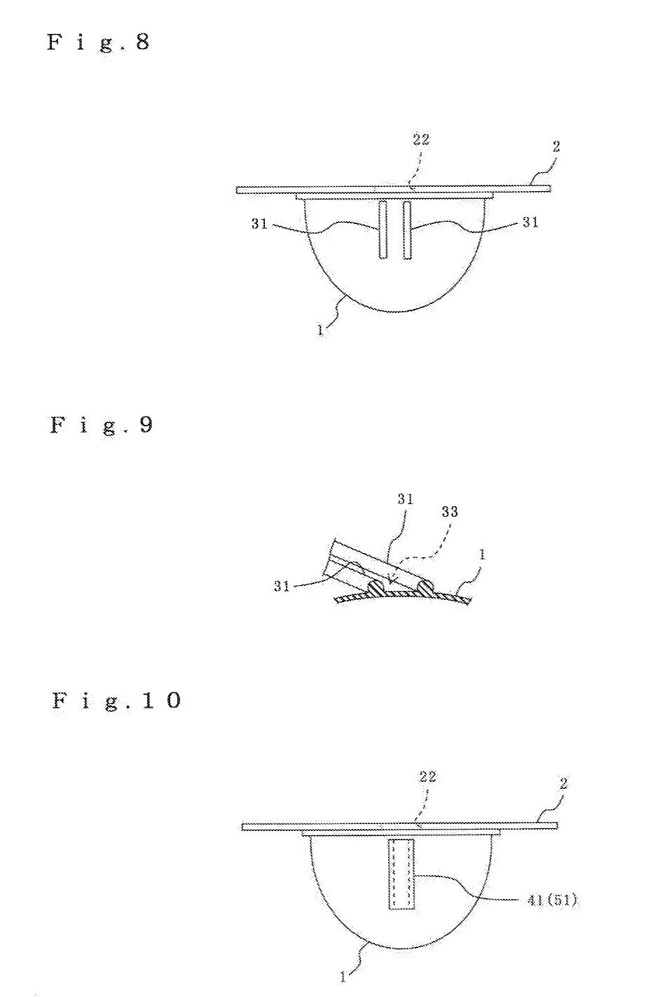

First of all, as shown in FIGS. 8 and 9, the gap forming member in another form may be, for example, a convex body protruding on an outer surface of the balloon dome part 1, the convex body formed by a series of wall-shaped bodies 31. These wall-shaped bodies 31 are arranged at predetermined intervals so as to be substantially parallel to each other, and the gap 33 may be formed continuously between the two wall-shaped bodies 31, 31.

The gap forming members constituted of the wall-shaped bodies 31 ensure a more stable continuous non-contact space between the two wall-shaped bodies 31, 31 as shown in FIG. 9, as compared to that formed by the partially protruding granular bodies. Accordingly, the non-contact space serves as the main gap 33 that surely guides blood flowing inside the parent blood vessel Bv to the branched blood vessel by. The blood flowing inside the parent blood vessel Bv flows into the branched blood vessel by through the flux hole 22 of the balloon plane part 2 and through the gap 33. Thus, the treatment for aneurysm An can be carried out without occluding the entrance to the branched blood vessel bv.

### Embodiment 5

The gap forming member in another form may be, for example, a convex body having a through hole formed on the outer surface of the balloon dome part 1, as shown in FIG. 10, the convex body being a tubular body 41 having a hollow part therein. The hollow part which the tubular body 41 originally has may serve as a continuously ensured gap 43.

The gap forming member constituted of the tubular body 41 more reliably ensures a continuous non-contact space with the hollow part of the tubular body 41 as shown in FIG. 11, as compared to the partially protruding granular bodies and the continuously protruding wall-shaped bodies. Accordingly, the non-contact space serves as the main gap 43 that surely guides blood flowing inside the parent blood vessel Bv to the branched blood vessel bv. The blood flowing inside the parent blood vessel Bv flows into the branched blood vessel bv through the flux hole 22 of the balloon plane part 2 and through the gap 43. Thus, the treatment for aneurysm An can be carried out without occluding the entrance to the branched blood vessel bv.

### Embodiment 6

An example of another form may be a convex body having a through hole formed on the outer surface of the balloon dome part 1, as shown in FIG. 10, the convex body being a U-shaped body 51 which forms a hollow part between an inner surface thereof and the outer surface of the balloon dome part 1. An inner part covered by the inner surface of the U-shaped body 51 may serve as a continuously ensured gap 53.

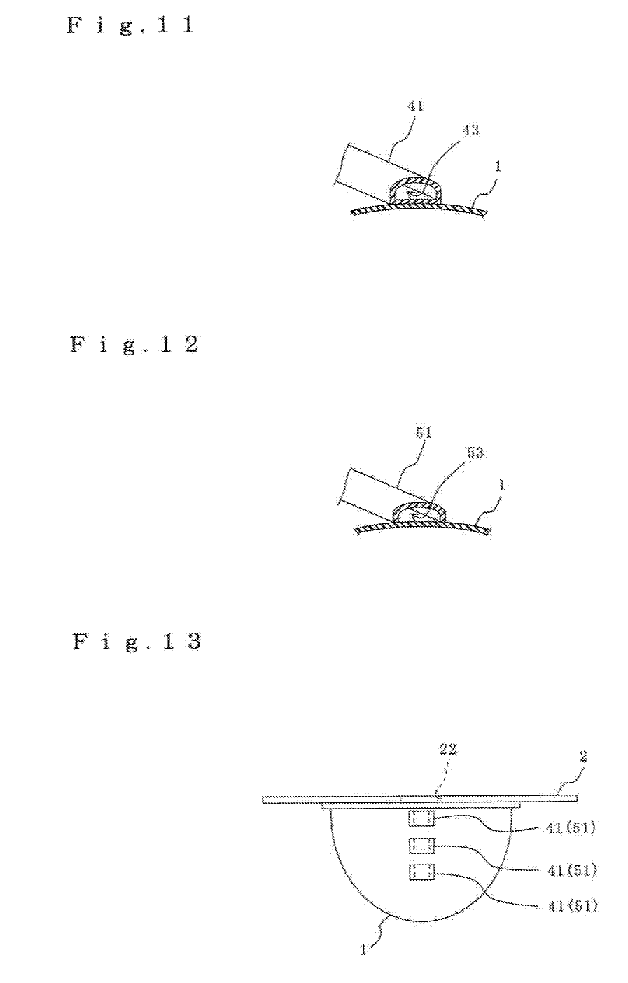

In the gap forming member constituted of the U-shaped body 51, a hollow part formed by a recess part (concave part) formed on the inner surface of the U-shaped body 51 substantially serves as a through hole as shown in FIG. 12. Accordingly, and the gap forming member more reliably ensures a continuous non-contact space, as compared to the partially protruding granular bodies and the series of protruding wall-shaped bodies. Thus, the non-contact space serves as the main gap 53 that surely guides blood flowing inside the parent blood vessel Bv to the branched blood vessel bv. The blood flowing inside the parent blood vessel Bv flows into the branched blood vessel by through the flux hole 22 of the balloon plane part 2 and through the gap 53. Thus, the treatment for aneurysm An can be carried out without occluding the entrance to the branched blood vessel bv.

### Embodiment 7

Furthermore, in the above-described fifth and sixth embodiments, the gap forming members may be partially provided on the outer surface of the balloon dome part 1. Specifically, as shown in FIG. 13, multiple of such gap forming members may be placed at desired intervals in a manner that the resultant gaps 43 of the tubular bodies 41 or the resultant gaps 53 of the U-shaped bodies 51 are oriented in the same direction. FIG. 13 shows that three of such tubular bodies 41 or U-shaped bodies 51 are provided.

As mentioned above, when multiples of the tubular bodies 41 or U-shaped bodies 51 are placed at desired intervals, a portion of the outer surface of the balloon dome part 1 can be exposed between the tubular bodies 41 or between the U-shaped bodies 51 while the gaps 43 or 53 are surely formed.

In this way, when the balloon dome part is folded to be housed in a lumen of a protective outer sheath member in the preparation of operating the aneurysm embolization device, the balloon dome part 1 can be made smaller (thinner) without creating bulkiness due to the gap forming members, resulting in easiness in folding the balloon dome part 1.

Next, description will be given of methods for operating the aneurysm embolization devices with the above-mentioned constitutions.

The aneurysm embolization devices of the present invention can be used for different shapes and sites of an aneurysm as appropriate. Here, an operation method in which an aneurysm is occluded using an aneurysm embolization device 30 shown in the above-described fourth embodiment will be described as an example.

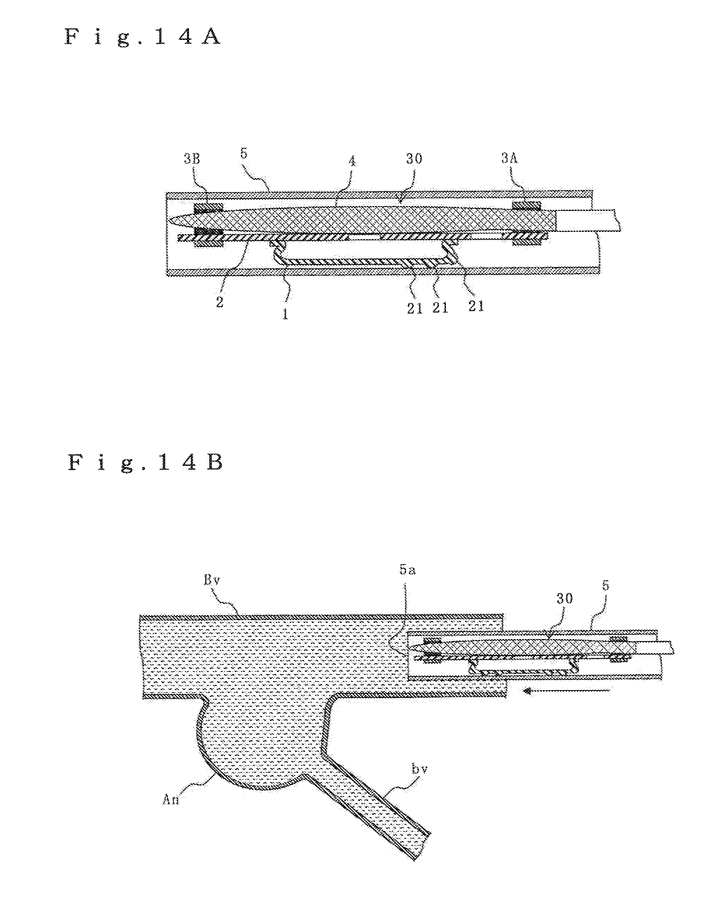

FIGS. 14A to 17B are process sectional views for sequentially illustrating the method for operating the aneurysm embolization device 30 shown in the third embodiment described above.

Firstly, as shown in FIG. 14A, the aneurysm embolization device 30 according to the third embodiment is folded to be housed in a lumen of a protective outer sheath member 5 that is formed of a flexible elongated-body having the lumen therein. This aneurysm embolization device 30 includes a balloon dome part 1 and a balloon plane part 2. The balloon plane part 2 includes an influx hole 12 and a flux hole 22. The balloon dome part 1 includes a gap forming member 21. The balloon plane part 2 further includes a stent 3 (3A and 3B) which is formed of an approximately tubular body and radially expands in a blood vessel, and an expansion balloon 4 which is expandable inside the stent 3.

It is preferred that such a protective outer sheath member 5 be flexible so that the member can easily follow meanderings of a blood vessel. The protective outer sheath member 5 may be a microcatheter used for cerebrovascular treatment. The microcatheter may be a wire-guided catheter which follows a guide wire having been inserted in a blood vessel ahead of the wire-guided catheter, and which is mainly used for aneurysm embolization. Alternatively, the microcatheter may be a flow-guided catheter which is guided by bloodstream. Examples of materials used for such a catheter include: various thermoplastic resins and thermosetting resins including polyvinyl chloride and polyolefins such as polyethylene, polypropylene, polyurethane, ethylene-propylene copolymers and ethylene-vinyl acetate copolymers, polyesters such as polyethylene terephthalate and polybutylene terephthalate, polyamide, polyimide, polytetrafluoroethylene, polyvinylidene-fluoride, and other fluorocarbon resins; thermoplastic elastomers such as polyamide elastomer and polyester elastomer; various rubbers; and the like.

Meanwhile, it is desired that that the protective outer sheath member (microcatheter) 5, at least a tip part thereof, be formed of a radiopaque material including metals such as platinum, gold, silver and tungsten, and alloys thereof, and that the protective outer sheath member 5 have an imaging ability allowing to monitor the manipulation using radioscopy. This enables a location of the tip part to be monitored during the operation of the protective outer sheath member 5.

Then, as shown in FIG. 14B, the protective outer sheath member 5 housing the aneurysm embolization device 30 is inserted into a parent blood vessel Bv, and the protective outer sheath member 5 is manipulated to place a tip 5a of the protective outer sheath member 5 at a desired position where the aneurysm embolization device 30 is intended to be left. In other words, the tip 5a of the protective outer sheath member 5 is placed in the vicinity of an aneurysm An having a branched blood vessel bv inside. As mentioned above, the protective outer sheath member 5 thus provided can transport the aneurysm embolization device 30 to the vicinity of the aneurysm while housing the aneurysm embolization device 30.

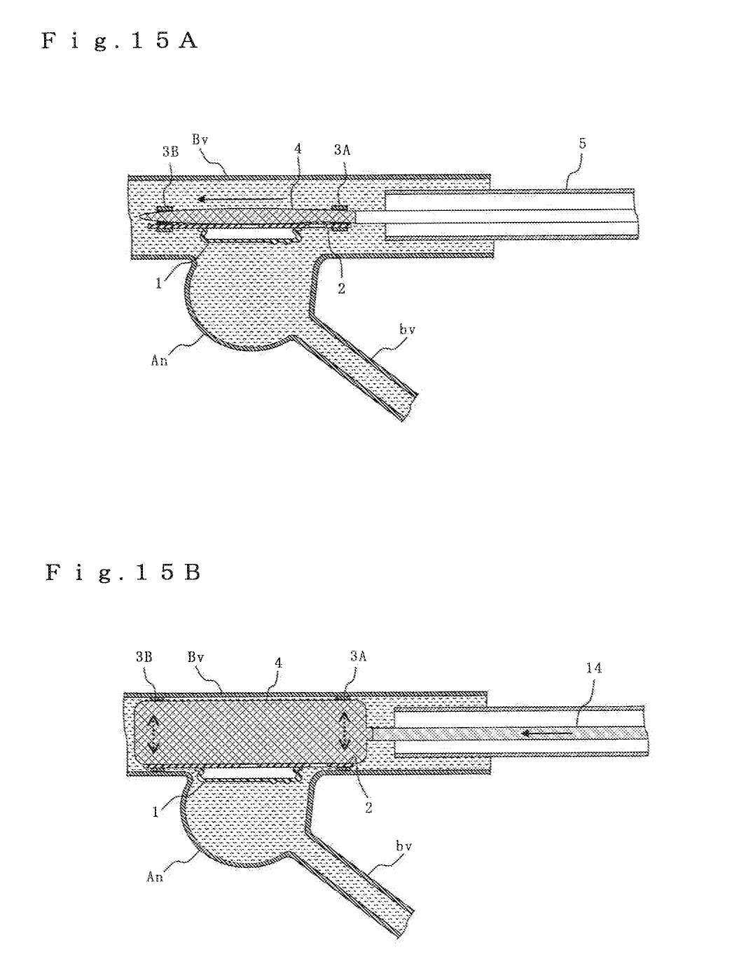

Subsequently, as shown in FIG. 15A, the aneurysm embolization device 30 is ejected from the tip 5a of the protective outer sheath member 5, and delivered above the aneurysm An. An arrow in the figure indicates a direction in which the aneurysm embolization device 30 is ejected from the protective outer sheath member 5.

Then, as shown in FIG. 15B, a liquid, for example, isotonic saline is supplied through a liquid-supplying tube 14 to the aneurysm embolization device 30, while the position of the aneurysm embolization device 30 is maintained. The liquid causes the expansion balloon 4 to expand, and then the stent 3 (3A, 3B) to expand. The stent 3 (3A, 3B) is bonded by pressure to the parent blood vessel Bv, thereby placing the balloon dome part 1 in the aneurysm An while deploying the balloon plane part 2 so as to cover a mouth part of the aneurysm An. A solid arrow in the figure indicates a direction in which the liquid (saline) is supplied through the liquid-supplying tube 14, and dotted arrows indicate directions in which the stent 3 (3A, 3B) is expanded by inflation of the expansion balloon 4.

As mentioned above, the balloon plane part 2 can be placed in the parent blood vessel so as to occlude the aneurysm by providing the expansion balloon 4. Thus, the aneurysm embolization device 30 inserts the balloon dome part 1 into the aneurysm An, and is left so that the balloon plane part 2 can cover the mouth part of the aneurysm An.

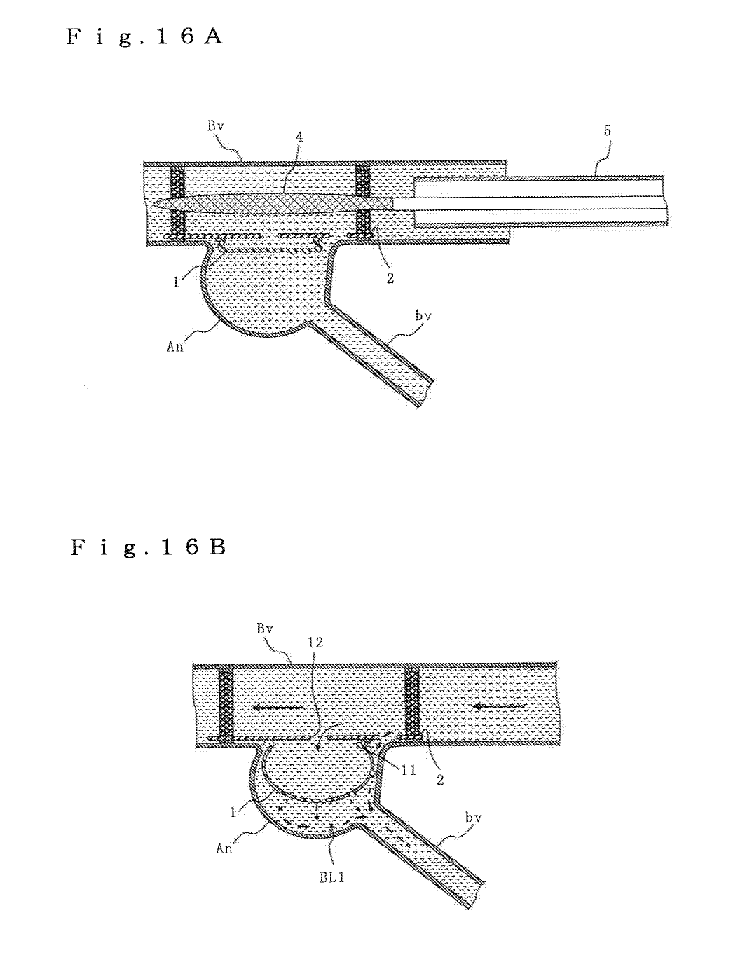

Subsequently, after the aneurysm embolization device 50 is stabilized in a blood vessel by the expansion of the stent 3 (3A and 3B), the expansion balloon 4 is deflated as shown in FIG. 16A. The expansion balloon 4 is then slid back from the inside of the stent 3 and removed from the inside of the parent vessel Bv together with the protective outer sheath member 5.

In the above-described manner, the aneurysm embolization device 30 according to the third embodiment of the present invention is operated.

As a result, as shown in FIG. 16B, blood streaming in the parent vessel Bv flows into the balloon dome part 1 from an influx hole 12 of the balloon plane part 2. This blood (hereinafter, referred to as “blood in the balloon dome part”) BL2 expands the balloon dome part 1, and pushes blood having been in the aneurysm An (i.e., a small amount of blood remained at the inner wall of the aneurysm An and the outside of the balloon dome part 1; hereinafter, referred to as “blood in the aneurysm”) BL1 out of the aneurysm An, that is, to the side of the branched blood vessel bv. Note that, in the figure, a thick solid arrow indicates a direction in which blood flows inside the parent blood vessel Bv; a thin solid arrow indicates a direction in which blood flows from the parent blood vessel Bv into the balloon dome part 1; and a thick dotted arrow indicates a direction in which blood flows from the parent blood vessel Bv through the aneurysm An into the branched blood vessel bv. A thin dotted arrow indicates a direction in which the balloon dome part 1 expands.

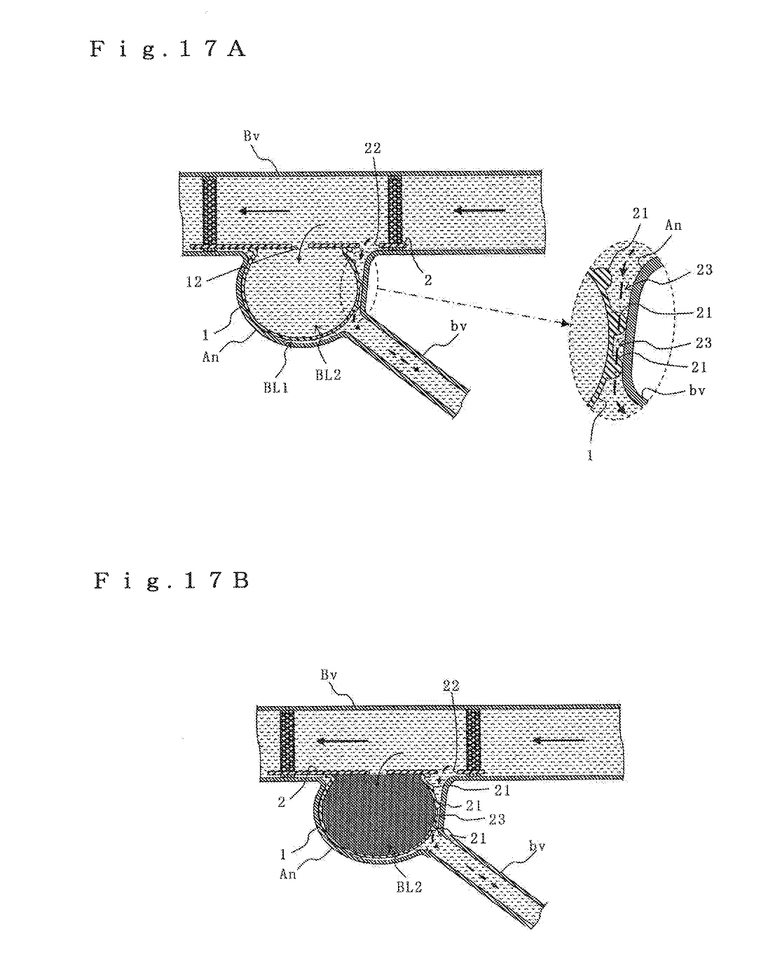

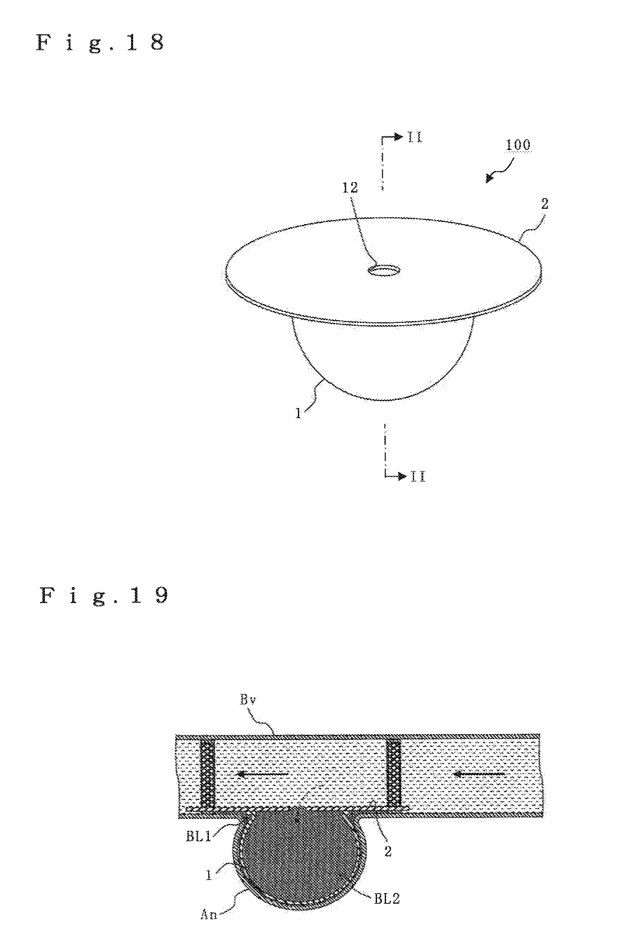

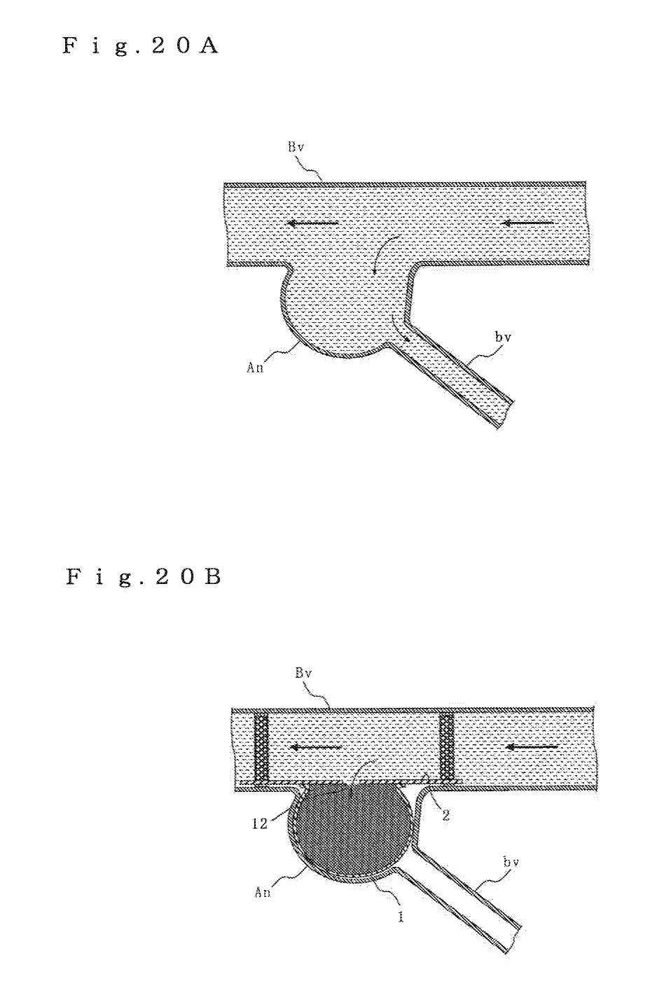

Subsequently, once the inside of the balloon dome part 1 is filled with the flowed blood as shown in FIG. 17A, the stream of the blood in the balloon dome part BL2 is repressed. During this event, gap forming members 21 provided to the balloon dome part 1 come into contact with the inner surface of the aneurysm An, as the balloon dome part 1 expands. As shown in the partially-enlarged view of a region surrounded by a dotted ellipse in FIG. 17A (see also FIG. 4), a gap 23 is formed between the outer surface of the balloon dome part 1 and the inner surface of the aneurysm An so as to ensure the blood flow to the entrance to the branched blood vessel bv and to prevent the balloon dome part 1 from occluding the entrance to the branched blood vessel bv. As mentioned above, blood flowing inside the parent blood vessel Bv flows into the branched blood vessel bv through the flux hole 22 of the balloon plane part 2 and then through the gap 23. A thick dotted arrow in the figure indicates a direction in which blood flows from the parent blood vessel Bv through the inside of the aneurysm An into the branched blood vessel bv.

Then, as shown in FIG. 17B, the blood BL2 inside the balloon dome part whose stream has been repressed forms a clot. Therefore, the treatment can be carried out while occluding the aneurysm An, and the blood flowing inside the parent blood vessel Bv can be securely flow into the branched blood vessel bv by the flux hole 22 of the balloon plane part 2 and by the gap 23 formed by the gap forming members 21 provided to the balloon dome part 1. Note that since the blood BL1 inside the aneurysm eventually forms a clot where it is not flowing, there is no risk of rupturing the aneurysm An.

In the above-described manner, by using the aneurysm embolization device according to the present invention, it is possible to occlude an aneurysm, even when there is a branched blood vessel inside of the aneurysm, readily and reliably in an appropriate manner according to the shape of a neck part of the aneurysm and the size thereof, and also to treat the aneurysm without occluding the entrance of the branched blood vessel.

### INDUSTRIAL APPLICABILITY

The present invention is industrially useful in the field where aneurysm embolization devices are handled to treat an aneurysm in a blood vessel. The invention is particularly useful for a market of aneurysm embolization devices to treat an aneurysm in the brain by occluding the aneurysm.

1. An aneurysm embolization device for treating an aneurysm in a blood vessel by occluding the aneurysm, comprising at least:
a sac-shaped balloon dome part which is inserted into the aneurysm, then expanded and left therein; and
a flexible sheet-shaped balloon plane part which is provided at an opening of the balloon dome part and covers a mouth part of the aneurysm, wherein
the balloon plane part includes: a first hole communicating with an inside of the balloon dome part; and a second hole communicating with an outside of the balloon dome part, and
the balloon dome part includes a gap forming member which forms a desired non-contact space, where an outer surface of the balloon dome part does not come into contact with an inner surface of the aneurysm, when the balloon dome part is expanded in the aneurysm.

2. The aneurysm embolization device according to claim 1, wherein a diameter of the opening of the balloon dome part is larger than a diameter of the first hole of the balloon plane part.

3. The aneurysm embolization device according to claim 1, wherein the balloon plane part further includes an anchor member (stent) which is expanded and left in the blood vessel.

4. The aneurysm embolization device according to claim 3, wherein the anchor member is attached to at least one end of the balloon plane part.

5. The aneurysm embolization device according to claim 1, wherein the gap forming member is a plurality of convex bodies protruding on the outer surface of the balloon dome part.

6. The aneurysm embolization device according to claim 5, wherein the convex bodies are granular bodies arranged at desired intervals.

7. The aneurysm embolization device according to claim 5, wherein the convex bodies are at least two wall-shaped bodies arranged substantially parallel to each other at a desired interval.

8. The aneurysm embolization device according to claim 1, wherein the gap forming member is a tubular body which has a through hole therein and is placed on the outer surface of the balloon dome part.

9. The aneurysm embolization device according to claim 1, wherein the gap forming member is a U-shaped body having a recess part on an inner surface thereof, the U-shaped body forming a through hole with the outer surface of the balloon dome part and the recess part when the gap forming member is placed on the outer surface of the balloon dome part.

10. The aneurysm embolization device according to claim 2, wherein the balloon plane part further includes an anchor member (stent) which is expanded and left in the blood vessel.

11. The aneurysm embolization device according to claim 10, wherein the anchor member is attached to at least one end of the balloon plane part.

12. The aneurysm embolization device according to claim 11, wherein the gap forming member is a plurality of convex bodies protruding on the outer surface of the balloon dome part.

13. The aneurysm embolization device according to claim 10, wherein the gap forming member is a plurality of convex bodies protruding on the outer surface of the balloon dome part.

14. The aneurysm embolization device according to claim 4, wherein the gap forming member is a plurality of convex bodies protruding on the outer surface of the balloon dome part.

15. The aneurysm embolization device according to claim 3, wherein the gap forming member is a plurality of convex bodies protruding on the outer surface of the balloon dome part.

16. The aneurysm embolization device according to claim 2, wherein the gap forming member is a plurality of convex bodies protruding on the outer surface of the balloon dome part.

17. The aneurysm embolization device according to claim 16, wherein the convex bodies are granular bodies arranged at desired intervals.

18. The aneurysm embolization device according to claim 15, wherein the convex bodies are granular bodies arranged at desired intervals.

19. The aneurysm embolization device according to claim 14, wherein the convex bodies are granular bodies arranged at desired intervals.

20. The aneurysm embolization device according to claim 13, wherein the convex bodies are granular bodies arranged at desired intervals.
